(19) United States
(12) Patent Application Publication
(10) Pub. No.: US 2023/0304083 A1
(43) Pub. Date: Sep. 28, 2023

(54) Methods and Systems of Producing Single Stranded DNA via PCR Using Biotin-Labeled Primers

(71) Applicant: Applied DNA Sciences, Inc., Stony Brook, NY (US)

(72) Inventors: Yuhua Sun, Stony Brook, NY (US); Ming-Hwa Liang, Stony Brook, NY (US)

(21) Appl. No.: 18/095,785

(22) Filed: Jan. 11, 2023

Related U.S. Application Data
(60) Provisional application No. 63/299,684, filed on Jan. 14, 2022.

(51) Int. Cl. C12Q 1/686; C12Q 1/6853

(52) U.S. Cl. CPC C12Q 1/686; C12Q 1/6853

(57) ABSTRACT

The present invention provides methods of producing single-stranded DNA (ssDNA) from a double-stranded DNA (dsDNA) template. The method includes selecting either the (+)-strand or the (−)-strand of a dsDNA template as a target ssDNA, wherein if the target ssDNA is the (+)-strand of the dsDNA template, a biotin label is added onto the reverse primer, and wherein if the target ssDNA is the (−)-strand of the dsDNA template, a biotin label is added onto the forward primer. Next PCR is performed to produce biotinylated dsDNA fragments. Once the PCR is terminated, the biotinylated dsDNA fragments are purified. Next the target ssDNA is separated from the biotinylated dsDNA by immobilizing the biotinylated dsDNA onto a surface and precipitating the target ssDNA.

### CROSS-REFERENCE TO RELATED APPLICATION

This application claims benefit of U.S. Provisional Patent Application No. 63/299,684, filed Jan. 14, 2022, the disclosure of which is incorporated herein by reference in its entirety.

### TECHNICAL FIELD

The present invention relates to systems and methods to produce single-stranded DNA (ssDNA) manufactured via polymerase chain reactions (PCR).

### BACKGROUND OF THE INVENTION

The manufacture of large quantities of high-quality ssDNA is currently a major bottleneck in mRNA production and rAAV manufacture utilized in, among other things, gene therapy and vaccines. Currently, bacterial plasmids, which are small circular episomal DNA molecules that can replicate independently of bacterial chromosomal DNA, are utilized as the primary source of DNA to produce vectors. In addition to long amplification times, measured in days or weeks, the amplification of DNA via bacterial plasmids for use in viral vector manufacture has additional drawbacks such as the necessity of complex and expensive purification steps, the risk of endotoxin contamination, antibiotic resistance gene transfer, other plasmid derived DNA sequence transfers, as well as challenges with integration into robotic and/or automated workflows.

### SUMMARY OF THE INVENTION

The present invention are methods of producing single-stranded DNA (ssDNA) from a double-stranded DNA (dsDNA) template. The method comprises: (a) providing a dsDNA template; (b) selecting either the (+)-strand or the (−)-strand of the dsDNA template as a target ssDNA; (c) providing forward primers and reverse primers, wherein if the target ssDNA is the (+)-strand of the dsDNA template, a biotin label is added onto the reverse primer, and wherein if the target ssDNA is the (−)-strand of the dsDNA template, a biotin label is added onto the forward primer; (d) performing a polymerase chain reaction (PCR) comprising denaturing of the dsDNA template, annealing the forward primers and the reverse primers to the template, extending the primers using a thermostable DNA polymerase, repeating several rounds of the PCR, wherein biotinylated dsDNA fragments are produced. Once the PCR is terminated, the biotinylated dsDNA fragments are purified, and (f) the target ssDNA is separated from the biotinylated dsDNA, wherein separating comprises: (i) immobilizing the biotinylated dsDNA onto a surface, (ii) adding a basic solution to the immobilized biotinylated dsDNA to form a dsDNA-exposed solution, (iii) neutralizing the dsDNA-exposed solution, and (iv) precipitating the target ssDNA.

### BRIEF DESCRIPTION OF THE DRAWINGS

FIG. 1: Bioanalyzer® printout showing dsDNA with ITRs with one Biotin label shown as a clean peak.

FIG. 2: Gel Image of dsDNA and ssDNA. After denaturation and Biotin purification, the non-labeled strand was collected and a clear ssDNA band was observed on the gel.

FIG. 3: Schematic depiction of the production and isolation of ssDNA.

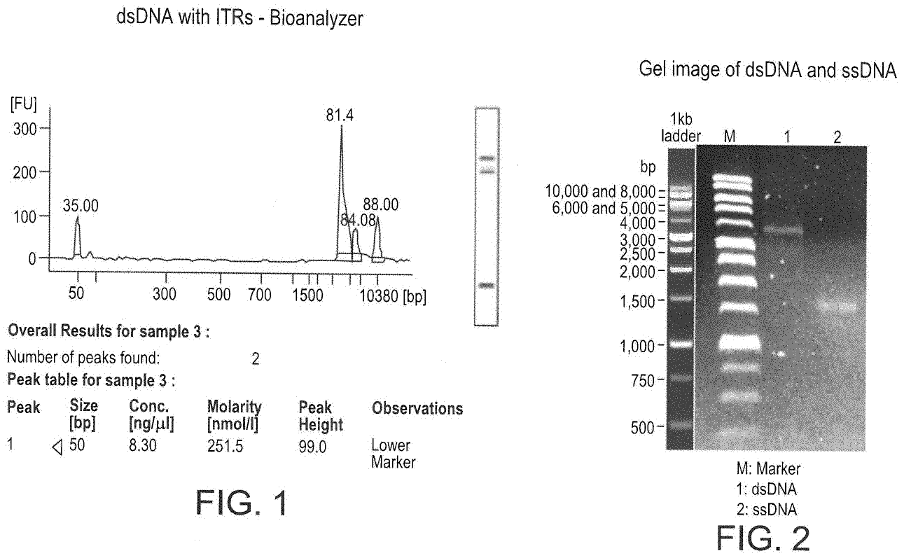

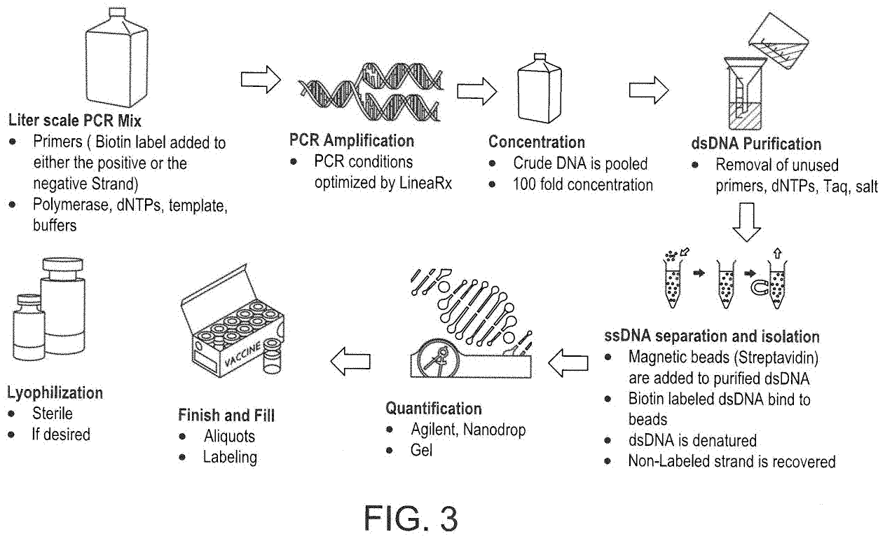

### DETAILED DESCRIPTION OF THE INVENTION

The present invention relates to methods of producing single-stranded DNA (ssDNA) via the Polymerase Chain Reaction (PCR). Single-stranded DNA is important for many biotherapeutic applications, including mRNA production and rAAV manufacture. The methods of the invention comprise using selective biotin labeled primers in PCR and subsequent denaturation and filtration of ssDNA.

The polymerase chain reaction (PCR) is based on three steps: (1) denaturation of a double stranded DNA (dsDNA) template into single strands; (2) annealing of primers (i.e., forward and reverse primers) to a target region on each original strand for new strand synthesis; and (3) extension of the new DNA strands from the primers using thermostable DNA polymerase (e.g., Taq polymerase). Several rounds of these steps are performed. The PCR results in the synthesis of defined portions of the original DNA sequence, i.e., the target DNA.

Positive sense and negative sense DNA refer to the coding sequence and non-coding sequence of the dsDNA template, respectively. In particular, if a DNA sequence directly gives the same mRNA sequence from the transcription, it is known as (+)-strand or sense-DNA. If a DNA sequence produces complementary mRNA sequence from the transcription, it is known as (−)-strand or antisense-DNA. Forward primers (i.e., 5′ primers) anneal to the (−)-strand of the dsDNA, which runs from 3′ to 5′ direction, whereas reverse primers (i.e., 3′ primers) anneal to the (+)-strand of the dsDNA, which runs from 5′ to 3′ direction.

In the methods of the invention, either the (+)-strand or the (−)-strand of the dsDNA template is selected as a target ssDNA. If the target ssDNA is the (+)-strand, a biotin label is added onto the reverse primer; and if the target ssDNA is the (−)-strand, a biotin label is added onto the forward primer.

In some embodiments, in order to inhibit steric hindrance and facilitate the binding between a biotin label and an immobilization surface (e.g., streptavidin beads), a spacer molecule is placed between the biotin label and an oligonucleotide of a primer. The spacer molecule forms long, flexible linker arms between the biotin label and the oligonucleotide. Typically, a spacer molecule is at least six carbon atoms in length. An example of a spacer molecule is a C3 Spacer phosphoramidite. Multiple C3 Spacers can be added, as needed (e.g., about one to about twenty C3 Spacers), to introduce a long hydrophilic spacer arm. Another example of a spacer molecule is a Spacer 9, which is a triethylene glycol chain that is 9 atoms in length (i.e., 6 carbons and 3 oxygens). A further example of a spacer molecule is Spacer 18, which is an 18-atom hexa-ethyleneglycol.

In the methods of the invention, PCR is performed using the DNA template and the aforementioned selectively biotinylated primers. For example, large scale PCR proceeds with optimized conditions for best yield and the highest quality. Once the PCR is terminated, the biotinylated dsDNA fragments are purified. In particular, the PCR product is purified to remove, for example, unused primers, deoxyribose nucleotide triphosphate (dNTP), salt and Taq polymerase. Purification methods may include, but are not limited to, centrifugal device for concentration, size exclusion (SEC) chromatography, anion exchange chromatography, and/or alcohol precipitation. Next, the purified dsDNA can be characterized by various analytical methods such as, for example, Thermo Scientific NanoDrop®, Bioanalyzer®, and High Performance Liquid Chromatography (HPLC). Together with Bioanalyzer software and reagents, the 2100 Bioanalyzer instrument provides electrodriven flow and separation of biomolecules through interconnected networks of microchannels.

After purification of the dsDNA, the biotinylated dsDNA is denatured to obtain target non-labeled ssDNA. Such denaturation proceeds as follows. First, the biotinylated dsDNA is immobilized onto a surface with methods known in the art. For example, the biotinylated dsDNA can be immobilized onto uniform and superparamagnetic beads about 0.5 to 2 μm in diameter (e.g., 1 μm in diameter), with a monolayer of streptavidin covalently coupled to the surface and preferably further blocked with Bovine Serum Albumin (BSA). The monolayer of streptavidin leaves the vast majority of the biotin binding sites sterically available for binding of the biotinylated dsDNA. For example, the biotinylated dsDNA can be immobilized with streptavidin coupled Dynabeads™ kilobaseBINDER™ Kit (Thermo Fisher, Cat #60101). The Dynabeads® kilobaseBINDER™ Kit is designed for immobilizing long double stranded DNA molecules (>2 kb). There is a very high binding affinity of the streptavidin-biotin interaction (K_{d}=10⁻¹⁵). The amount of biotinylated dsDNA immobilized will depend on fragment size. Typically, the immobilization is effected at a ratio of about 200 μg of dsDNA to about 1 mg of Dynabeads™. As would be known to a skilled artisan, one would follow manufacturer's user manual and scale up the volume accordingly. Next a basic solution is added to the immobilized dsDNA (e.g., dsDNA bound beads). For example, about 1 mL of 1M freshly prepared NaOH solution is added to the immobilized dsDNA (e.g., dsDNA bound beads) to form a dsDNA-exposed solution. The dsDNA is exposed to the basic solution for at least about 8 to 12 minutes (e.g., about 10 minutes) at about room temperature. Next, the dsDNA-exposed solution is collected and is neutralized with an equal molar acid (e.g., acetic acid) and diluted (e.g., diluted at least about 5 fold). Next, the ssDNA is precipitated by methods known in the art. For example, ethanol is added to precipitate the target ssDNA.

After the target ssDNA is precipitated, the results are analyzed, e.g., a gel is loaded for analysis. For example, urea gel analysis of dsDNA and ssDNA can be accomplished as described in Nucleic Acid Research (2009) 37(17):e112. doi:10.1093/nar/gkp539. In the methods of the invention, long ssDNA is produced, typically, greater than about 1, about 2, about 3, about 4 kilobase (kb), with a maximum of about 5 kb.

In some embodiments, the methods of the present invention include producing ssDNA without flanking ITRs. Single stranded DNA without flanking inverted terminal repeats (ITRs) is relevant for mRNA production. In such embodiments, conventional PCR is used. In some embodiments, the primer lengths are between about 18 to 25 base pairs. In some embodiments, PCR is performed as a series of three steps: (i) denaturing step at about 98° C.; (ii) annealing step at between about 55° C. to 65° C.; and (iii) extension step at between about 70° C. to 73° C. In some embodiments, the maximum difference between the melting temperatures of both primers is about 5° C. In some embodiments, the GC content of the primers is between about 40 and 60%, with the 3′ of a primer ending in C or G to promote binding. Primers should not contain regions forming secondary structures, e.g., self-dimers/hairpins and primer-dimer formation.

In some embodiments, the methods of the present invention include producing ssDNA with flanking ITR regions. Flanking ITRs are relevant for rAAV production. In such embodiments, a modified PCR is preferred due to the secondary structures of the ITR regions that are ill-suited for conventional PCR. In such embodiments, the PCR amplification disclosed in U.S. Patent Publication No. 2021/0054384 are used. The disclosure of U.S. Patent Publication No. 2021/0054384 is incorporated herein by reference in its entirety.

### Example

Manufacturing of long (>2 kb) ssDNA—ITR flanked
1. PCR amplification of target dsDNA
   a. Primer design: If the target ssDNA is the (+)-strand, add a biotin label on the reverse primer, and vice versa for the (−)-strand.
   b. Optimize PCR conditions for best yield and the highest quality
   c. Large scale PCR production of biotinylated dsDNA
2. Biotinylated dsDNA fragment purification
   a. The PCR product is purified to get rid of the unused primers, dNTPs, salt and Taq polymerase. Purification method may include but not limited to centrifugal device for concentration, SEC chromatography and or anion exchange chromatography, alcohol precipitation etc.
   b. Purified dsDNA is characterized by various analytical methods such as Nanodrop, Bioanalyzer, HPLC etc.
3. Separation of the biotinylated dsDNA to obtain target non-labeled ssDNA
   a. Immobilize the biotinylated dsDNA with the streptavidin coupled Dynabeads™ kilobaseBINDER™ Kit (Thermo Fisher, Cat #60101) at a ratio of 200 ug of dsDNA to 1 mg of Dynabeads, follow manufacturer's user manual and scale up the volume accordingly.
   b. To separate the dsDNA to ssDNA, add 1 mL of 1M freshly prepared NaOH to the DNA bound beads to dissociate the non-biotinylated strand from the beads at room temperature for at least 10 min, collect the liquid for next step.
   c. Neutralized the solution with equal molar of acetic acid, dilute at least 5 folds before loading the gel for analysis
   d. Ethanol precipitates the ssDNA for final analysis
4. Urea gel analysis of dsDNA and ssDNA (Nucleic Acid Research, 2009, vol. 37, No. 17, e112. doi:10.1093/nar/gkp539)
   a. GelRed analysis: 1% Agarose gel with 1M Urea was freshly prepared, Load the denatured and non-denatured product onto the gel, GelRed dye, 1×TAE running buffer, 55V for 3 hours. (Denature: 80° C. for 5 min, quick chill on ice)
5. Final finish and fill based on desired condition.

Although the invention has been described with reference to the above examples and embodiments, it is not intended that such references be constructed as limitations upon the scope of this invention.

1. A method of producing single-stranded DNA (ssDNA) from a double-stranded DNA (dsDNA) template, the method comprising:
(a) providing a dsDNA template;
(b) selecting either the (+)-strand or the (−)-strand of the dsDNA template as a target ssDNA;
(c) providing forward primers and reverse primers, wherein if the target ssDNA is the (+)-strand of the dsDNA template, a biotin label is added onto the reverse primer, and wherein if the target ssDNA is the (−)-strand of the dsDNA template, a biotin label is added onto the forward primer;
(d) performing a polymerase chain reaction (PCR) comprising denaturing of the dsDNA template, annealing the forward primers and the reverse primers to the template, extending the primers using a thermostable DNA polymerase, repeating several rounds of the PCR, wherein biotinylated dsDNA fragments are produced;
(e) once the PCR is terminated, purifying the biotinylated dsDNA fragments; and
(f) separating the target ssDNA from the biotinylated dsDNA, wherein separating comprises: (i) immobilizing the biotinylated dsDNA onto a surface, (ii) adding a basic solution to the immobilized biotinylated dsDNA to form a dsDNA-exposed solution, (iii) neutralizing the dsDNA-exposed solution, and (iv) precipitating the target ssDNA,
wherein ssDNA is produced.

2. The method of claim 1, wherein the produced ssDNA is about 1 to about 4 kilobases in length.

3. The method of claim 1, wherein the ssDNA comprises flanking ITR regions.

4. The method of claim 1, wherein the ssDNA does not comprise flanking ITR regions.

5. The method of claim 1, wherein purification comprises removing unused primers, deoxyribose nucleotide triphosphate (dNTP), salt and Taq polymerase.

6. The method of claim 5 wherein purification comprises centrifugal concentration, SEC chromatography, anion exchange chromatography, and/or alcohol precipitation.

7. The method of claim 1 wherein immobilizing surface comprises streptavidin coupled Dynabeads™.

8. The method of claim 1 wherein the basic solution is about 1 mL of 1M NaOH solution.

9. The method of claim 8 wherein the basic solution is neutralized with an equal molar acetic acid.

10. The method of claim 1 wherein the ssDNA is precipitated by the addition of ethanol.

11. The method of claim 1, wherein a spacer molecule is between the biotin label and an oligonucleotide of the primer.

12. The method of claim 11, wherein the spacer molecule is at least six carbon atoms in length.

13. The method of claim 11, wherein the spacer molecule is a phosphoramidite.

14. The method of claim 11, wherein the spacer molecule is a triethylene glycol chain.

15. The method of claim 11, wherein the spacer molecule is an 18-atom hexa-ethyleneglycol.
